(19) United States
(12) Patent Application Publication
(10) Pub. No.: US 2026/0224881 A1
(43) Pub. Date: Aug. 6, 2026

(54) IMPLANTABLE BONE-CONDUCTION ACTUATOR

(71) Applicant: BrioHealth Solutions, Inc., Burlington, MA (US)

(72) Inventors: Gill Brent Bearnson, Salt Lake City, UT (US); Scott D. Miles, Sandy, UT (US)

(73) Assignee: BrioHealth Solutions, Inc., Burlington, MA (US)

(21) Appl. No.: 19/045,842

(22) Filed: Feb. 5, 2025

(51) Int. Cl. A61M 60/178; A61M 60/876

(52) U.S. Cl. CPC A61M 60/178; A61M 60/876; A61M 2205/18

(57) ABSTRACT

The present invention relates to a system for conveying status information of a mechanical circulatory support in a subject, the system comprising an actuator implanted in the subject, and a mechanical circulatory support controller, wherein the actuator is communicably coupled to the mechanical circulatory support controller, and the actuator is not coupled to the subject's cochlea.

### BACKGROUND OF THE INVENTION

Medical devices that are adapted for implantation often have high power requirements, and must remain either connected to an power source external to a body, or must frequently be recharged or otherwise serviced. Patients who have such implanted medical devices typically rely on these devices for important health interventions, such as, for example, maintaining a healthy heartbeat or correcting an unhealthy heartbeat or inadequate cardiac blood flow. If the device were to deactivate or malfunction without notice, this could present a serious threat to the patient.

### SUMMARY OF THE INVENTION

The present invention relates broadly to systems, devices, and methods for detecting an alert state in an implanted cardiac device in a subject, and alerting the subject of such alert state.

In an embodiment, the present invention relates to a system for detecting a fault state in a mechanical circulatory support in a subject, the system comprises an actuator implanted in the subject, and a mechanical circulatory support controller, wherein the actuator is communicably coupled to the mechanical circulatory support controller, provided that the actuator is not attached to the subject's cochlea.

In an embodiment, the actuator may be implanted internally in the subject and attached to a bone of the subject. In an embodiment, exemplary human bones to which the actuator may be attached may be selected from a group consisting of: sternum, rib, clavicle, scapula, humerus, radius, ulna, cranium, mandible, femur, tibia, fibula, or a combination thereof. In an embodiment, the bone may be the subject's sternum.

In an embodiment, the mechanical circulatory support is selected from a group consisting of: a fully implantable left ventricular assist system and a mostly implantable left ventricular assist system.

In an embodiment, the surface of the actuator may include features that promote ingrowth or on-growth of the bone into or onto the actuator surface over time, thus improving coupling of energy transmission into the bone. In an embodiment, the actuator may include features that promote ingrowth of the bone into the actuator surface. In an embodiment, the features that promote ingrowth of the bone into the actuator surface over time may include porous surface features. In an embodiment, the features that promote ingrowth of the bone into the actuator surface may include surfaces which may comprise pores having a pore size of about 10 microns to about 400 microns. In an embodiment, the surfaces comprising pores may be composed of titanium, titanium felt, hydroxyapatite, bone cement, fabric, felt, expanded polytetrafluoroethylene, cast polymer, cast urethane, urethane foam, or a combination thereof. In an embodiment, the cast polymer is a cast porous polymer. In an embodiment, the actuator may be affixed to the bone with a bone screw. In an embodiment, the actuator may be composed of titanium.

In an embodiment, the actuator may be communicably coupled to a mechanical circulatory support controller via a cable or via radio frequency (RF) transmission. In an embodiment, the actuator may be a bone conduction speaker. In an embodiment, when the mechanical circulatory support controller determines that the mechanical circulatory support is in an alarm state, the actuator may impart haptic sensation to the subject. In an embodiment, the haptic sensation may be transmitted mechanical energy, such as a vibration, which is transmitted via bone. The alarm state may be a fault state, a battery charge status, low battery charge and/or mechanical circulatory support malfunction.

In an embodiment, the actuator may be programmed to activate and deactivate in response to status data received from the mechanical circulatory support controller.

In another embodiment, the present invention provides a mechanical circulatory support fault-state alert device, the device comprising an implantable actuator that is configured to be attached to a non-cochlear bone of a patient, and the surface of the actuator may include features that promote ingrowth or on-growth of the non-cochlear bone into or onto the actuator surface and improves transfer of energy into the non-cochlear bone. In an embodiment, the features that promote ingrowth or on-growth of the bone into or onto the actuator surface may include porous surface features. In an embodiment, the porous surfaces may have a size of about 10 microns to about 400 microns. In an embodiment, the features that promote ingrowth of the bone into the actuator surface over time may be made from material selected from: titanium, titanium felt, hydroxyapatite, bone cement, fabric, felt, expanded polytetrafluoroethylene, cast polymer, cast urethane, urethane foam, or a combination thereof. In an embodiment, the cast polymer may be a cast porous polymer. In an embodiment, the features that promote ingrowth of the bone into the actuator surface over time may be made from titanium.

In an embodiment, the implantable actuator may be communicably couplable to the mechanical circulatory support controller via a cable or by radio frequency communication.

In an embodiment, the non-cochlear bone may be selected from the group consisting of: sternum, rib, clavicle, scapula, humerus, radius, ulna, cranium, mandible, femur, tibia, fibula, or a combination thereof. In an embodiment of the device, the non-cochlear bone may be sternum.

In an embodiment, the actuator may be a bone-conduction haptic actuator. In an embodiment, the bone-conduction haptic actuator may comprise a hermetically sealed titanium can.

In an embodiment, the mechanical circulatory support may be a fully implantable left ventricular assist system or a mostly implantable left ventricular assist system.

In an embodiment, when the mechanical circulatory support is detected to be in a fault state, the actuator may impart mechanical energy on the patient's non-cochlear bone. In an embodiment, the mechanical circulatory support fault state may be selected from the group consisting of: a battery charge status, a low battery charge, and a malfunction. In an embodiment, the mechanical circulatory support fault state may be low battery.

In still another embodiment, the present invention provides a method of alerting a mechanical circulatory support patient to a mechanical circulatory support status, the method comprising implanting a system for detecting an alert state in a mechanical circulatory support in a subject, the system comprising an actuator implanted in the patient and attached to a bone of the patient, and a mechanical circulatory support controller, wherein the actuator is communicably coupled to the mechanical circulatory support controller, provided however that the actuator is not attached to the subject's cochlea; wherein the system imparts a haptic sensation on the patient via bone conduction when the mechanical circulatory support controller determines that the mechanical circulatory support is in an alert state.

In an embodiment, the bone may be selected from the group consisting of: sternum, rib, clavicle, scapula, humerus, radius, ulna, cranium, mandible, femur, tibia, fibula, or a combination thereof. In an embodiment of the method, the bone may be the patient's sternum.

In an embodiment, the actuator may be affixed to the bone with a bone screw.

In an embodiment, the actuator may be affixed to the bone with a clamp.

In an embodiment, the actuator may be affixed to the bone with a fixation device.

In an embodiment, the fixation device may be shaped such that the fixation device may receive two adjacent ribs.

In an embodiment, the mechanical circulatory support may be selected from the group consisting of: a fully implantable left ventricular assist system and a mostly implantable left ventricular assist system.

In an embodiment, the actuator may be communicably coupled to the mechanical circulatory support controller via a cable or by radio frequency communication.

In an embodiment, the alert state may be a battery charge status. In an embodiment, the alert state is selected from the group consisting of: low battery charge and mechanical circulatory support malfunction.

In an embodiment, the actuator may comprise a hermetically sealed titanium can.

### BRIEF DESCRIPTION OF DRAWINGS

For the purpose of facilitating an understanding of the subject matter sought to be protected, there are illustrated in the accompanying drawings embodiments thereof, from an inspection of which, when considered in connection with the following description, the subject matter sought to be protected, its construction and operation, and many of its advantages should be readily understood and appreciated.

FIG. 1 shows a schematic of an actuator assembly in accordance with embodiments disclosed herein.

FIG. 2 shows a side view of the schematic of FIG. 1.

FIG. 3 shows a cross-sectional view of an actuator assembly attached to rib bones in accordance with embodiments disclosed herein.

FIG. 4 shows a side view of the actuator assembly of FIG. 3.

FIG. 5 shows a schematic of the actuator assembly mounted to a human sternum in accordance with embodiments disclosed herein.

### DETAILED DESCRIPTION OF THE INVENTION

While the present invention is susceptible of embodiments in many different forms, there is shown in the drawings, and will herein be described in detail, embodiments of the invention, including a preferred embodiment, with the understanding that the present disclosure is to be considered as an exemplification of the principles of the present invention and is not intended to limit the broad aspect of the invention to any one or more embodiments illustrated herein. As used herein, the term “present invention” is not intended to limit the scope of the claimed invention, but is instead used to discuss exemplary embodiments of the invention for explanatory purposes only.

Patients with heart problems sometimes receive cardiac implantable electronic devices to manage and/or monitor patients' heart conditions. Examples of cardiac implantable electronic devices include, without limitation, pacemakers, implantable cardioverter defibrillators (ICDs), biventricular pacemakers, cardiac loop recorders, and mechanical circulatory support (MCS) devices. Examples of MCS devices include, without limitation, temporary and permanent MCSs. MCSs include left ventricular assist device (LVADs). MCSs may also be classified as fully implantable left ventricular assist systems (FILVAS) or mostly implantable left ventricular assist systems (MILVAS). Other cardiac assist devices or replacements are also envisioned. For example, the device might be a Right Ventricular Assist Device (RVAD) or a Total Artificial Heart (TAH).

Conditions and uses for which an MCS may be indicated include heart failure, bridge-to-transplant (i.e., circulatory support for a patient who is awaiting heart transplantation), support for unstable angina or irregular heart rhythm (arrhythmia), recovery from an acute cardiac event such as a myocardial infarction, myocarditis, cardiogenic shock, post-cardiotomy shock, post-surgical recovery (i.e., temporary mechanical cardiovascular support to maintain blood flow after a complex medical procedure), and other conditions, such as post-partum cardiomyopathy. Patients with an MCS usually have a medical condition and would be at high risk of complication, severe bodily harm, or even death, if the prescribed MCS were to malfunction or fail.

MCS patients who are implanted with a FILVAS or MILVAS usually connect their device to an external power source. Many MCS devices are designed to be able to disconnect entirely from external power and monitoring. For example, the system may operate entirely from an implanted battery. To disconnect completely, patients might choose to also disconnect from any external monitoring device that can provide status information about the state of charge of the implanted battery or any other alarm or alert condition that is relevant to patient.

The systems, devices, and methods of the present invention are intended to allow the implanted system to communicate to the patient through an actuator that is coupled to a bone inside the patient's body. Thus, the present invention relates broadly to systems, devices, and methods for detecting an alert state in an implanted cardiac device in a subject, and then providing an alerting to the subject.

In an embodiment, the present invention relates to a system for detecting a fault state in a mechanical circulatory support in a subject, the system comprising an actuator implanted in the subject, and a mechanical circulatory support controller, wherein the actuator is communicably coupled to the mechanical circulatory support controller, provided however that the actuator is not attached to the subject's cochlea.

One feature of the systems, devices, and methods of the present invention is use of bone conduction haptics. For example, bone conduction headphones that couple an actuator to a skull bone near the ear from outside the body are commercially available. Such headphones are known to allow certain hearing-impaired people to better “hear” sound, depending on the cause of hearing loss. For patients with severe hearing loss, such as, e.g., cochlear damage, bone coupled actuators do not allow them to hear speech, but they can still feel the sensation of vibration.

The systems, devices, and methods of the present invention use haptic tactile sensation such as vibration to allow an MCS patient to feel the sensation from inside the body through an actuator that is coupled to a bone other than a cochlear bone. According to the present invention, an actuator may be attached to a bone and communicably coupled to the patient's internal MCS controller, e.g., via a cable. The surface of the actuator may contain features that promote ingrowth or on-growth of the bone into or onto the actuator, which improves efficiency of transmission of mechanical energy into the bone. The selected bone could be the sternum or another bone in the region of the implant that is convenient for connection, such as a rib or clavicle.

Thus, In an embodiment, the actuator implanted in the subject may be attached to a bone of the subject. In an embodiment, the bone may be selected from the group consisting of: sternum, rib, clavicle, scapula, humerus, radius, ulna, cranium, mandible, femur, tibia, fibula, or a combination thereof. Alternately, the actuator could be implanted in compliant tissue other than bone, but the actuator in such case would have its energy dissipated unless it were a very high power actuator, and the patient would be unlikely to feel the sensation of the actuator's haptic alert function. Coupling the actuator to a non-cochlear bone, however, provides a significant improvement of the patient's ability to feel vibrations, relative to compliant soft tissue, that an alert condition in the MCS system, such as low battery or other error, needs to be addressed.

In an embodiment, the mechanical circulatory support is selected from the group consisting of a fully implantable left ventricular assist system and a mostly implantable left ventricular assist system.

In an embodiment, the surface of the actuator may include features that promote ingrowth or on-growth of the bone into or onto the actuator and that improve transfer of vibration into the bone. In an embodiment, the features that promote ingrowth of the bone into the actuator surface may comprise porous surface features. In an embodiment, the pores of the porous surface may have a pore size of about 10 microns to about 400 microns. The surface features may also comprise folds or ridges.

The pores may be any suitable shape, including, for example, circle, semicircle, conical, triangular, corpuscle, crescent, cup, cylindrical, ellipse, oval, heart, lunar, semi-ellipse, semi-oval, hemispherical, ring-shaped, spiked, or any combination thereof. The pores may be any suitable size, e.g., from 1 micron to 10,000 microns. For instance, the pores may be about 5 micron to about 1,000 microns. In embodiments, as noted above, the pores may have a pore size of about 10 microns to about 400 microns or about 20 microns to about 300 microns or about 30 microns to about 200 microns.

Surface coatings may also be used to promote ingrowth or on-growth or both. Examples of a suitable surface coating material include titanium plasma spray coating. In an embodiment, the surfaces comprising pores may include titanium, hydroxyapatite, bone cement, fabric, felt, expanded polytetrafluoroethylene, cast polymer, cast urethane, urethane foam, or a combination thereof. In embodiments, the surface features may include sintered beads. In an embodiment, the cast polymer is a cast porous polymer. The surface feature, such as sintered beads, may include any suitable material, including, e.g., titanium. In an embodiment, the actuator may be affixed to the bone with a bone screw and/or with bone cement. The bone cement may be polymethyl methacrylate (PMMA), calcium phosphate cement (CPC), glass polyalkenoate cement (GPC), or any combination thereof.

In an embodiment, the bone may be cleaned prior to affixing the actuator to the bone surface. In an embodiment wherein the actuator is affixed using bone cement, the cement may be cured for 10 to 15 minutes. In an embodiment, curing may be photocuring. Persons skilled in the art will understand that bone cements' curing processes will vary depending on the cement material used and certain ambient conditions such as temperature and humidity. For example, PMMA is typically formed by the mixture of a liquid monomer and powdered polymer, which commences a reaction that turns the liquid component into a solid. Some CPCs may be light-cured CPCs, which include a photocurable resin phase which is cured by exposure to electromagnetic radiation, such as, e.g., ultraviolet radiation. Some GPCs may be cured by an acid-base reaction initiated by a practitioner mixing two or more components. Some GPCs comprise one or more photocurable monomers, and are cured by exposure to electromagnetic radiation, such as, e.g., ultraviolet radiation. Other GPCs may comprise a mixture of monomers that cure by acid-base reaction as well as by photocuring.

Generally, the implant should be fixedly coupled during the bone ingrowth/on-growth period, or else osteoblast formation will be disrupted and not succeed in the ingrowth/on-growth process. Some bone-to-implant relative movement, on the order of microns for example, is allowable, and may in fact promote ingrowth/on-growth. Without intending to be bound by any particular theory, if the movement is too great or the bone-to-implant distance is too great, the osteoblasts cannot successfully bridge the gap and form an appropriate bone-to-implant attachment. Preferably, the actuator-to-bone gap upon implantation would be less than about 800 microns. In some embodiments, upon implantation, a force or load may be placed on the actuator until the ingrowth/on-growth process has completed.

Fixation of the implant to a bone during the ingrowth period may require a screw attachment, bone cement, fixation device, clamp, or similar. Where a screw attachment is used, the need for an ingrowth/on-growth coating or substrate may be obviated. Any means of holding the implant against a bone surface without much movement, including use of a bone screw, will promote ingrowth/on-growth. When affixed by means such as a bone screw, ingrowth/on-growth would not be necessary to hold the actuator fixed near the surface of the bone, because the actuator may efficiently transmit mechanical forces (e.g., vibrations) to the bone, thereby generating a sufficient haptic sensation in the patient.

In an embodiment of a fixation device, the actuator may be fitted with a U-shaped “shoe” on opposite sides. Ingrowth-prone surface texture or features may be provided in the interior of the U-shaped features. The ribs may be spread, the actuator inserted, and the ribs then allowed to return back to their normal spacing. In returning to their original position, two adjacent ribs may drop into the U-shaped features on the actuator, thus holding it in place. Tissue and bone ingrowth could then occur over time.

In an embodiment, the actuator may comprise titanium. More particularly, in an embodiment, the actuator may be coupled to an interior of a hermetically sealed can. The hermetically sealed can be made from any suitable material, such as titanium. In embodiments, the hermetically sealed can may have thin walls, which vibrate in response to the coupled actuator.

In an embodiment, the actuator may be communicably coupled to the mechanical circulatory support controller via a cable. In an embodiment, the actuator may be a bone conduction speaker. In an embodiment, when the mechanical circulatory support controller determines that the mechanical circulatory support is in an alarm state, the actuator may impart haptic sensation (e.g., vibration) on the subject. In an embodiment, the haptic sensation may be transmitted mechanical energy, which is transmitted through bone. In an embodiment, the actuator may be included in a sternal transcutaneous connector, which serves to connect external electronics to the internal MCS system.

In an embodiment, the alarm state may be a fault state, a battery charge status, or other status of the implant. In an embodiment, the alarm state is selected from the group consisting of: low battery charge and mechanical circulatory support malfunction. In an embodiment, the alarm state may be low battery charge. In an embodiment, the alarm state may be malfunction. The malfunction may be, e.g., a MCS device mechanical failure.

In an embodiment, the frequency, number or types of haptic sensation may provide information to the user. For example, the urgency and frequency of the haptic feedback may increase if the low battery is more serious. For example, the when battery charge level is low, e.g., between 10% and 20%, the actuator may impart a haptic sensation into the patient's bone every 60 seconds. When the battery charge level is very low, e.g., between 5% and 10%, the actuator may impart a haptic sensation into the patient's bone every 20 seconds. When the battery charge level is low, e.g., between 1% and 5%, the actuator may impart a haptic sensation into the patient's bone constantly. Likewise, if there is a system fault, the actuator may provide a preset number of haptic sensations to the patient, thus providing the patient with a “code” to assess the fault or other communication of the implant.

In an embodiment, the actuator may be programmed to activate and deactivate in response to status data received from the mechanical circulatory support controller.

In another embodiment, the present haptic sensation provides a mechanical circulatory support fault-state alert device, the device comprising an implantable actuator, wherein the implantable actuator is configured to be attached to a non-cochlear bone of a patient, and wherein the surface of the actuator includes features that promote ingrowth or on-growth of the non-cochlear bone into or onto the actuator surface over time and improves coupling of energy into the non-cochlear bone. In an embodiment, the features that promote ingrowth or on-growth of the bone into or onto the actuator surface over time may comprise porous surface features of a size of about 10 microns to about 400 microns. In an embodiment, the features that promote ingrowth of the bone into the actuator surface over time may be made from material selected from; titanium, hydroxyapatite, bone cement, fabric, felt, expanded polytetrafluoroethylene, cast urethane, urethane foam, or a combination thereof. In an embodiment, a textured, porous ingrowth surface may be created by sintering titanium beads onto a surface. In an embodiment, these beads may be in the range of 50 to 150 microns in diameter which, when sintered together, will form interstitial voids with a very irregular shape of a range of sizes, thereby forming the textured surface.

The pores may be any suitable shape, including circle, semicircle, conical, triangular, corpuscle, crescent, cup, cylindrical, ellipse, oval, heart, lunar, semi-ellipse, semi-oval, hemispherical, ring-shaped, spiked, or any combination thereof.

In an embodiment, the implantable actuator may be communicably couplable to the mechanical circulatory support controller via a cable.

In an embodiment, the non-cochlear bone may be selected from the group consisting of: sternum, rib, clavicle, scapula, humerus, radius, ulna, cranium, mandible, femur, tibia, fibula, or a combination thereof. In an embodiment of the device, the non-cochlear bone may be sternum.

In an embodiment, the actuator may be a bone-conduction haptic actuator. In an embodiment, the bone-conduction haptic actuator may comprise a hermetically sealed titanium can.

In an embodiment, the mechanical circulatory support may be a fully implantable left ventricular assist system or a mostly implantable left ventricular assist system.

In an embodiment, when the mechanical circulatory support is detected to be in a fault state, the actuator may impart mechanical energy on the patient's non-cochlear bone. In an embodiment, the mechanical circulatory support fault state may be selected from the group consisting of: a battery charge status, a low battery charge, and/or a malfunction.

In still another embodiment, the present invention provides a method of alerting a mechanical circulatory support patient to a mechanical circulatory support status, the method comprising implanting a system for detecting an alert state in a mechanical circulatory support in a subject, the system comprising an actuator implanted in the patient and attached to a bone of the patient, and a mechanical circulatory support controller, wherein the actuator is communicably coupled to the mechanical circulatory support controller, provided however that the actuator is not attached to the subject's cochlea; wherein the system imparts a haptic sensation on the patient via bone conduction when the mechanical circulatory support controller determines that the mechanical circulatory support is in an alert state.

In an embodiment, the bone may be selected from the group consisting of: sternum, rib, clavicle, scapula, humerus, radius, ulna, cranium, mandible, femur, tibia, fibula, or a combination thereof. In an embodiment of the method, the bone may be the patient's sternum.

In an embodiment, the actuator may be affixed to the bone with a bone screw.

In an embodiment, the mechanical circulatory support may be selected from the group consisting of: a fully implantable left ventricular assist system and a mostly implantable left ventricular assist system.

In an embodiment, the actuator may be communicably coupled to the mechanical circulatory support controller via a cable.

In an embodiment, the alert state may be a battery charge status, low battery charge and/or mechanical circulatory support malfunction.

In an embodiment, the actuator may comprise a hermetically sealed titanium can.

As used herein, the term “coupled” can mean any physical, electrical, magnetic, or other connection, either direct or indirect, between two components. The term “coupled” is not limited to a fixed direct coupling between two entities. For instance, a non-fixed direct coupling may include a radio-frequency (RF) connection to a remote haptic actuator.

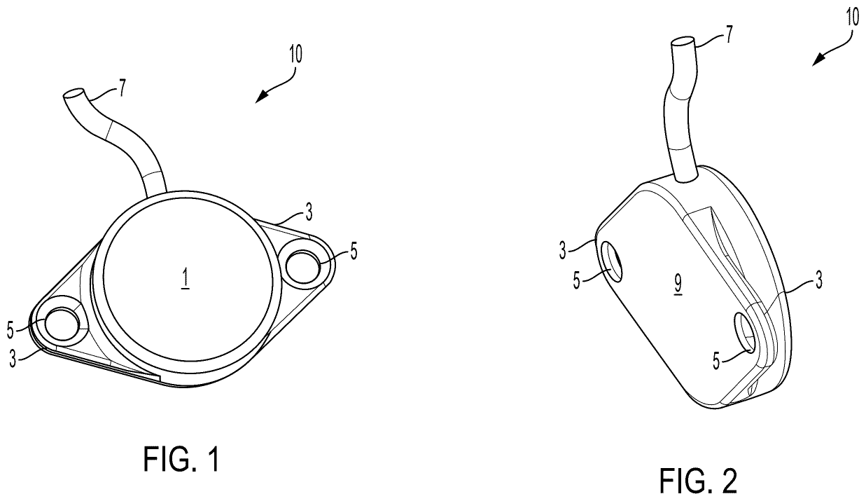

Referring now to FIG. 1, in an embodiment, the actuator assembly 10 includes the actuator 1 and mounting bracket 3. The mounting bracket 3 includes one or more apertures 5 configured to receive a fastener, such as a bone screw. The actuator 1 may be communicably coupled to the mechanical circulatory support controller via a cable 7. As shown in FIG. 2, the actuator surface 9 on the obverse side of the actuator assembly 10 may include features that promote ingrowth or on-growth of the bone into or onto the actuator surface, such as titanium, hydroxyapatite, bone cement, fabric, felt, expanded polytetrafluoroethylene, cast urethane, urethane foam, or a combination thereof.

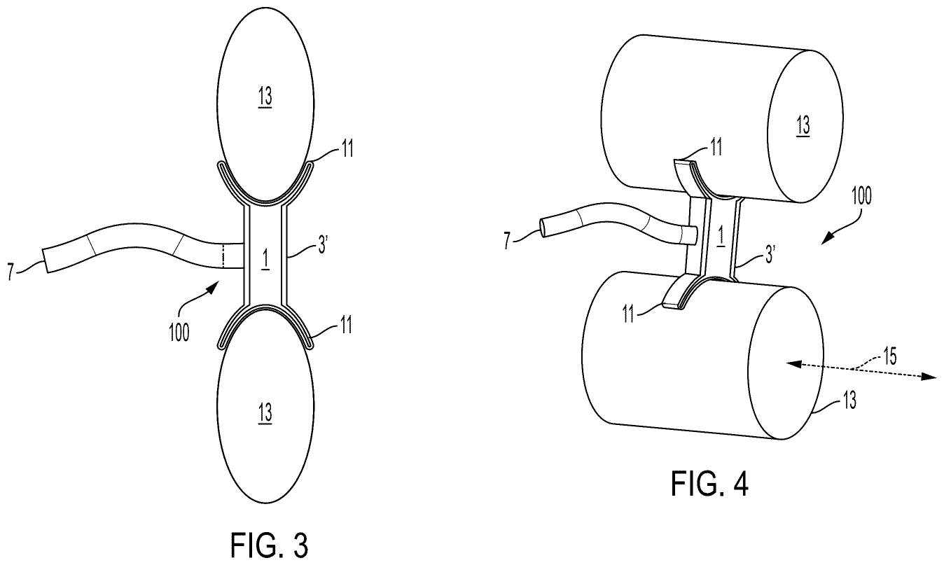

Referring now to FIG. 3, in another embodiment, the actuator assembly 100 may be configured to attach to the patient's ribs. The actuator assembly 100 includes actuator 1 and mounting bracket 3′. The mounting bracket 3′ includes U-shaped rib receptacles 11 on opposing sides of the mounting bracket 3′. These U-shaped rib receptacles 11 are so shaped as to fit around at least a portion of the patient's ribs, such as adjacent ribs 13 shown in FIG. 3. As best shown in FIG. 4, the actuator assembly 100 fits substantially orthogonal to a major axis 15 of, and between, the ribs 13. In this embodiment, if the U-shaped rib receptacles 11 have inner surfaces that are sufficiently textured, spiked, ribbed, or otherwise configured to increase friction between the U-shaped receptacles 11 and the bone of the ribs 13, it may be possible to exclude any other substance for attachment, such as bone cement or bone screws. However, the actuator assembly 100 could be installed in an inter-costal fixation manner. In such an application, the ribs would be spread apart and the device inserted in between. The ribs would then be allowed to collapse back onto the device which would nominally hold it in place. The surgeon may also choose to join together the two adjoining ribs on either side of the device using “umbilical tape” or similar. This would pull the ribs tightly against the device to prevent the ribs from spreading. The compression of the ribs against the device would also give the ribs an opportunity to grow into the fixation device, thus holding the device more solidly over time.

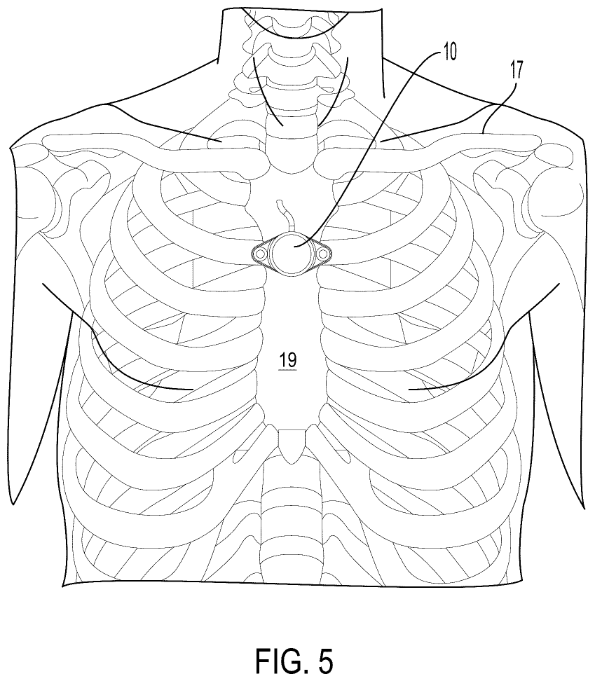

Referring to FIG. 5, the actuator assembly 10 may be mounted directly to the patient's skeletal system 17, such as to the sternum 19.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of the inventors' contribution. The actual scope of the protection sought is intended to be defined in the following claims when viewed in their proper perspective based on the prior art.

1. A system for conveying a fault state in a mechanical circulatory support in a patient, the system comprising:
an actuator implanted in the patient, and
a mechanical circulatory support controller,
wherein the actuator is communicably coupled to the mechanical circulatory support controller, and is coupled to a bone of the patient, but not coupled to a cochlea of the patient, and wherein the actuator is programmed to transfer energy to the bone of the patient in response to status data received from the mechanical circulatory support controller.

2. The system of claim 1, wherein the bone is selected from a group consisting of a sternum, a rib, clavicle, scapula, humerus, radius, ulna, cranium, mandible, femur, tibia, and a fibula of the patient.

3. The system of claim 1, wherein the bone is a sternum of the patient.

4. The system of claim 1, wherein the mechanical circulatory support is selected from a group consisting of a fully implantable left ventricular assist system and a mostly implantable left ventricular assist system.

5. The system of claim 1, wherein a surface of the actuator includes features that promote ingrowth or on-growth of the bone into or onto the actuator and is adapted to improve transfer of energy into the bone.

6. The system of claim 5, wherein the features include a porous surface.

7. The system of claim 6, wherein the porous surface includes pores having respective pore sizes of about 10 microns to about 400 microns.

8. The system of claim 6, wherein the porous surface includes pores composed of titanium, hydroxyapatite, bone cement, fabric, felt, expanded polytetrafluoroethylene, cast polymer, cast urethane, or urethane foam.

9. The system of claim 1, wherein the actuator is coupled to the bone with a bone screw, bone cement, fixation device, or a clamp.

10. The system of claim 1, wherein the energy transferred to the bone includes vibrations at a preset frequency and duration.

11. A mechanical circulatory support fault-state alert device, comprising:
an implantable actuator that is configured to be coupled to a non-cochlear bone of a patient, wherein a surface of the actuator includes porous surface features that promote ingrowth or on-growth of the non-cochlear bone into or onto the actuator and improves transfer of energy to the non-cochlear bone, the porous surface features include pores having respective pore sizes of about 10 microns to about 400 microns.

12. The device of claim 11, wherein the features are made from titanium, hydroxyapatite, bone cement, fabric, felt, expanded polytetrafluoroethylene, cast polymer, cast urethane, or urethane foam.

13. The device of claim 11, wherein the non-cochlear bone is selected from the group consisting of a sternum, a rib, clavicle, scapula, humerus, radius, ulna, cranium, mandible, femur, tibia, and a fibula of the subject.

14. The device of claim 11, wherein the non-cochlear bone is a sternum.

15. The system of claim 11, wherein the energy transferred to non-cochlear bone includes vibrations at a preset frequency and duration.

16. A method of providing a status of a mechanical circulatory support system to a patient, the method comprising:
implanting a system for detecting the status of the mechanical circulatory support system in the patient, the system including:
an actuator implanted in the subject and coupled to a bone of the patient, but not coupled to a cochlea of the patient, and
a mechanical circulatory support controller,
wherein the actuator is communicably coupled to the mechanical circulatory support controller, and the system imparts a vibration to the patient via bone conduction to convey the status of the mechanical circulatory system to the patient.

17. The method of claim 16, wherein the bone is selected from a group consisting of a sternum, a rib, clavicle, scapula, humerus, radius, ulna, cranium, mandible, femur, tibia, and a fibula of the patient.

18. The method of claim 16, wherein a surface of the actuator includes features that promote ingrowth or on-growth of the actuator into or onto the bone and improves transfer of energy to the bone, wherein the features include a porous surface having pores with respective pore sizes of about 10 microns to about 400 microns.

19. The method of claim 16, wherein the mechanical circulatory support is selected from a group consisting of a fully implantable left ventricular assist system and a mostly implantable left ventricular assist system.

20. The method of claim 16, wherein the status is a battery charge or fault alert.
